SEQUENCE LISTING (3 sequences)

SEQ ID NO: 1 - DNA, 1327 bp, Pseudomonas sp.
gatagagaggctgctgtagaatgcgcgcctcggttgagacgaaaggcttaaccaactgttctttaacaactgaatcaagcaattcgtgtgggtgcttgtgaggtaagactgatagtcaactgattatcagcatcacaaagcaacactcgttaattcgagagttacctttcattaatttgaaagttttgcgattgctgagccaagtttagggttttctcaaaacccaagcagtattgaactgaagagtttgatcatggctcagattgaacgctggcggcaggcctaacacatgcaagtcgagcggatgagaggagcttgctccttgatttagcggcggacgggtgagtaatgcctaggaatctgcctggtagtgggggataacgtccggaaacgggcgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggttccttgagaacttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtccc

SEQ ID NO: 2 - DNA, 18 bp, Artificial Sequence
  Synthetic
agtttgatcmtggctcag

SEQ ID NO: 3 - DNA, 16 bp, Artificial Sequence
  Synthetic
gacgggcggtgtgtrc

(19) United States
(12) Patent Application Publication
(10) Pub. No.: US 2022/0312775 A1
(43) Pub. Date: Oct. 6, 2022

(54) NEMATODE-ATTRACTING BACTERIA AND METHODS OF USING SAME

(71) Applicant: DEAD SEA AND ARAVA SCIENCE CENTER, D.N. Dead Sea (IL)

(72) Inventors: Amir SZITENBERG, Ein Gedi (IL); Rivka Shani ALEXANDER, Mitzpe Yericho (IL)

(21) Appl. No.: 17/640,912

(22) PCT Filed: Sep. 6, 2020
(86) PCT No.: PCT/IL2020/050969

§ 371 Date: Mar. 7, 2022

Related U.S. Application Data
(60) Provisional application No. 62/896,640, filed on Sep. 6, 2019.

(51) Int. Cl. A01N 63/27; C12N 1/20; A01P 19/00; A01M 1/02

(52) U.S. Cl. CPC A01N 63/27; C12N 1/205; A01P 19/00; A01M 1/02; C12R 2001/38

(57) ABSTRACT

The present invention is directed to a an isolated bacterial strain belonging to the genus of Pseudomonas having a nematode chemotaxis-inducing activity, a composition comprising same, and methods of using same, such as for attracting a nematode to a surface, or for protecting a plant from nematode-induced damage.

### CROSS-REFERENCE TO RELATED APPLICATIONS

The present application claims priority to U.S. Provisional Application No. 62/896,640, titled “NEMATODE-ATTRACTING BACTERIA AND METHODS OF USE THEREOF”, filed Sep. 6, 2019, the contents of which are incorporated herein by reference in their entirety.

### FIELD OF INVENTION

The present invention is in the field of biological control, and in some embodiments, is directed to the prevention and/or treatment of nematode-induced plant disease.

### BACKGROUND

Root knot nematodes (RKN) are among the world's most devastating plant pathogens, causing substantial yield losses in nearly all major agricultural crops. They are found in all regions that have mild winter temperatures and are regarded as one of the most serious threats to agriculture as climate change progresses. In their life-cycle, species of the Meloidogyne incognita group (MIG) hatch in the soil and invade a root. Once inside the roots, the worms form the characteristic knots from which their name arises. Each knot contains at least one nematode feeding from a unique cell-type (the giant cells), surrounded by a gall of dividing cortical cells. RKN interact with microorganisms throughout their life cycle, therefore providing opportunities for biological control.

Nematodes account for an estimated 14% of all worldwide plant losses, translated into ca. $90 billion dollars annually. Due to the controversial effect of nematicides and gradual phase-out of several prominent chemical nematicides in the last years including the ban of methyl bromide, there is still a great need for better generic, effective and non-hazardous nematicides.

### SUMMARY

The following embodiments and aspects thereof are described and illustrated in conjunction with systems, tools and methods which are meant to be exemplary and illustrative, not limiting in scope.

The present invention, in some embodiments thereof, is directed to a composition comprising bacteria having a nematode chemotaxis-inducing activity, and methods of use thereof, such as for attracting a nematode, and/or for preventing nematode-induced damage to plants.

According to a first aspect, there is provided an isolated bacterial strain of the genus Pseudomonas comprising a genome having at least 94% homology or identity to a genome deposited at NCBI under BioSample accession number SAMN15770455.

According to another aspect, there is provided a composition comprising the isolated bacterial strain of the invention and an agriculturally or environmentally acceptable carrier.

According to another aspect, there is provided a method for attracting a nematode to a surface, comprising contacting the surface with bacteria having nematode chemotaxis-inducing activity, thereby attracting the nematode to the surface.

According to another aspect, there is provided a method for protecting a plant from nematode-induced damage, comprising: contacting a growth medium comprising a nematode with a composition comprising bacteria having a nematode chemotaxis-inducing activity and an artificial support, wherein the bacteria is coupled to or within the artificial support, thereby protecting the plant from nematode-induced damage.

In some embodiments, the isolated bacterial strain comprises a genome having 100% homology or identity to the genome deposited at NCBI under a BioSample accession number SAMN15770455.

In some embodiments, the isolated bacterial strain comprises a polynucleotide sequence having at least 90% homology or identity to SEQ ID NO: 1.

In some embodiments, the isolated bacterial strain comprises a polynucleotide sequence having 100% homology or identity to SEQ ID NO: 1.

In some embodiments, the polynucleotide sequence having at least 90% homology or identity to SEQ ID NO: 1 is a 16S-rRNA sequence.

In some embodiments, the isolated bacterial strain is characterized by having a nematode chemotaxis-inducing activity, a nematode egg hatching inhibiting activity, a nematocidal activity, or any combination thereof.

In some embodiments, the composition further comprises an artificial support.

In some embodiments, the isolated bacterial strain is coupled to or within the artificial support.

In some embodiments, the artificial support is configured to trap a nematode attracted to the isolated bacterial strain.

In some embodiments, the nematode belongs to the genus Meloidogyne.

In some embodiments, the nematode is Meloidogyne incognita.

In some embodiments, the bacteria belong to the genus Pseudomonas.

In some embodiments, the bacteria is the isolated bacterial strain of the invention.

In some embodiments, the nematode is a plant parasitic nematode (PPN).

In some embodiments, the nematode belongs to the genus Meloidogyne.

In some embodiments, the composition is the composition of the invention.

In some embodiments, the composition is more attractant of the nematode than the root is attractant of the nematode.

In some embodiments, the composition is at least twice as attractant as the root.

In some embodiments, protecting the plant comprises reducing the penetration rate of the nematode to the root, reducing the number of nematodes penetrating the root, reducing the number of hatching eggs of the nematode, reducing the egg hatching rate of the nematode, or any combination thereof.

In some embodiments, the bacteria having nematode chemotaxis-inducing activity traps the nematode in the composition.

In some embodiments, the composition reduces the survival of the nematode.

In some embodiments, the composition kills the nematode.

In some embodiments, the composition has nematocidal activity.

In some embodiments, protecting comprises attracting the nematode to the composition.

In some embodiments, contacting the growth medium does not comprise contacting a root of the plant.

In some embodiments, contacting the growth medium is at a distance of at least 0.1 cm from a root of the plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C include an illustration and micrographs demonstrating an attraction assay. (1A) is a non-limiting illustration of an attraction assay set-up depicting a well of a 12-well plate comprising a root fragment incubated in 1 ml Pluronic-P127-Tris-MES gel, second stage juvenile larvae (J2) and a pipette tip (containing a bacterial isolate/sterile medium in control). (1B) is an image showing a root (arrow) and J2 larvae. (1C) is an image showing J2 larvae and a pipette tip (arrow) containing a bacterial isolate.

FIGS. 2A-2B include graphs showing the intra-specific genetic distance distribution based on (2A) P distances and (2B) tree-branch distances. (2A-2B) 318 sequences from 220 species yielding 213 intraspecies distances are included in the null distribution. The distance between the sequence of the herein disclosed isolate and its closest sequenced relative (●) is significantly larger than an intra-specific distance. (2A) P_{existing species}=0.0227; (2B) P_{existing species}=0.0045.

FIG. 3 includes a graph showing the distribution of hatchling counts after 24 and 48 hours, following four different treatments: (1) Biocasle capsule (Menashe and Kurzbaum, 2014) containing double distilled water (DDH); (2) Biocasle capsule containing the sterile medium; (3) Biocasle capsule containing the bacterial filtered medium (‘filtrate’); and (4) Biocasle capsule containing live bacteria in their medium.

### DETAILED DESCRIPTION

In some embodiments, the present invention is directed to a composition comprising isolated bacteria, wherein the bacteria has a nematode chemotaxis-inducing activity, that is to say a nematode attracting activity.

Methods of using the composition to attract a nematode and protect a plant from nematode-induced damage are also provided.

According to some embodiments, there is provided an isolated bacterial strain of the genus Pseudomonas.

In some embodiments, there is provided a composition comprising isolated bacteria, wherein at least 80% of the isolated bacteria is the isolated bacteria of the invention.

In some embodiments, there is provided a method of attracting a nematode to a surface, comprising contacting the surface with a composition of the invention.

In some embodiments, there is provided a method of protecting a plant comprising a root in a growth medium comprising a nematode from nematode-induced damage, comprising contacting the growth media comprising the nematode with a composition of the invention. The present invention is based, in part, on the surprising finding that bacteria isolated from eggplant root, which were identified as a novel species of Pseudomonas, attracted root knot nematode juvenile stage 2 larvae. This heretofore unknown species of bacteria was a stronger nematode attractant that eggplant root by more than an order of magnitude. Nematode larvae placed in a dish with both roots and the new bacteria were so strongly attracted to the bacteria that no worms migrated to the root. The novel bacteria may thus be used as a nematode trap to keep them away from plant roots that might otherwise have been harmed by invading nematodes.

### Bacteria Isolate

According to some embodiments, there is provided an isolated bacterial strain of the genus Pseudomonas comprising or characterized by having a genome with at least 94% homology or identity, 95% homology or identity, 96% homology or identity, 97% homology or identity, 98% homology or identity, at least 99% homology or identity, or 100% homology or identity to a genome deposited at NCBI under BioSample accession number SAMN15770455, or any value and range therebetween. Each possibility represents a separate embodiment of the invention.

According to some embodiments, the isolated bacterial strain (e.g., the bacteria of the invention) of the genus Pseudomonas comprises or is characterized by having a genome with 94-100% homology or identity, 95-99% homology or identity, 96-98% homology or identity, or 97-100% homology or identity to a genome deposited at NCBI under BioSample accession number SAMN15770455. Each possibility represents a separate embodiment of the invention.

In some embodiments, there is provided an isolated bacterial strain of the genus Pseudomonas comprising a genome comprising or consisting the genome deposited at NCBI under BioSample accession number SAMN15770455.

In some embodiments, the bacteria of the invention is isolated from a plant. In some embodiments, the bacteria is isolated from a root of a plant. In some embodiments, the bacteria is isolated from a sample comprising a root of a plant. In some embodiments, the bacteria is isolated from a sample comprising a root of a plant infested by nematodes. In some embodiments, the bacteria is isolated from a sample comprising soil in which a plant was or is cultured in. In one embodiment, a plant as used herein above, is an eggplant.

Methods for isolating bacteria are common, such as exemplified herein below, and would be apparent to one of ordinary skill in the art.

According to some embodiments, the isolated bacteria comprises a sequence with at least 80% homology or identity to

(SEQ ID NO: 1)
GATAGAGAGGCTGCTGTAGAATGCGCGCCTCGGTTGAGACGAAAGGCTTA
ACCAACTGTTCTTTAACAACTGAATCAAGCAATTCGTGTGGGTGCTTGTG
AGGTAAGACTGATAGTCAACTGATTATCAGCATCACAAAGCAACACTCGT
TAATTCGAGAGTTACCTTTCATTAATTTGAAAGTTTTGCGATTGCTGAGC
CAAGTTTAGGGTTTTCTCAAAACCCAAGCAGTATTGAACTGAAGAGTTTG
ATCATGGCTCAGATTGAACGCTGGCGGCAGGCCTAACACATGCAAGTCGA
GCGGATGAGAGGAGCTTGCTCCTTGATTTAGCGGCGGACGGGTGAGTAAT
GCCTAGGAATCTGCCTGGTAGTGGGGGATAACGTCCGGAAACGGGCGCTA
ATACCGCATACGTCCTACGGGAGAAAGCAGGGGACCTTCGGGCCTTGCGC
TATCAGATGAGCCTAGGTCGGATTAGCTAGTTGGTGAGGTAATGGCTCAC
CAAGGCGACGATCCGTAACTGGTCTGAGAGGATGATCAGTCACACTGGAA
CTGAGACACGGTCCAGACTCCTACGGGAGGCAGCAGTGGGGAATATTGGA
CAATGGGCGAAAGCCTGATCCAGCCATGCCGCGTGTGTGAAGAAGGTCTT
CGGATTGTAAAGCACTTTAAGTTGGGAGGAAGGGCATTAACCTAATACGT
TAGTGTTTTGACGTTACCGACAGAATAAGCACCGGCTAACTTCGTGCCAG
CAGCCGCGGTAATACGAAGGGTGCAAGCGTTAATCGGAATTACTGGGCGT
AAAGCGCGCGTAGGTGGTTCGTTAAGTTGGATGTGAAAGCCCCGGGCTCA
ACCTGGGAACTGCATCCAAAACTGGCGAGCTAGAGTACGGTAGAGGGTGG
TGGAATTTCCTGTGTAGCGGTGAAATGCGTAGATATAGGAAGGAACACCA
GTGGCGAAGGCGACCACCTGGACTGATACTGACACTGAGGTGCGAAAGCG
TGGGGAGCAAACAGGATTAGATACCCTGGTAGTCCACGCCGTAAACGATG
TCAACTAGCCGTTGGGTTCCTTGAGAACTTAGTGGCGCAGCTAACGCATT
AAGTTGACCGCCTGGGGAGTACGGCCGCAAGGTTAAAACTCAAATGAATT
GACGGGGGCCCGCACAAGCGGTGGAGCATGTGGTTTAATTCGAAGCAACG
CGAAGAACCTTACCTGGCCTTGACATGCTGAGAACTTTCCAGAGATGGAT
TGGTGCCTTCGGGAACTCAGACACAGGTGCTGCATGGCTGTCGTCAGCTC
GTGTCGTGAGATGTTGGGTTAAGTCCC.

In some embodiments, a polynucleotide sequence having at least 80% homology or identity to SEQ ID NO:1 is a 16S-ribosomal RNA (16S-rRNA) sequence. In some embodiments, SEQ ID NO: 1 is a fragment of a 16S-rRNA. In some embodiments, SEQ ID NO: 1 corresponds to bases 46-395 of a 16S-rRNA in the isolated bacteria of the invention. In some embodiments, at least 70% of the isolated bacteria of the invention comprise a polynucleotide sequence having at least 80% homology or identity to SEQ ID NO: 1. In some embodiments, at least 80% of the isolated bacteria of the invention comprise a polynucleotide sequence having at least 80% homology or identity to SEQ ID NO: 1. In some embodiments, at least 90% of the isolated bacteria of the invention comprise a polynucleotide sequence having at least 80% homology or identity to SEQ ID NO: 1. In some embodiments, at least 95% of the isolated bacteria of the invention comprise a polynucleotide sequence having at least 80% homology or identity to SEQ ID NO: 1. In some embodiments, at least 99% of the isolated bacteria of the invention comprise a polynucleotide sequence having at least 80% homology or identity to SEQ ID NO: 1. In some embodiments, 100% of the isolated bacteria of the invention comprise a polynucleotide sequence having at least 80% homology or identity to SEQ ID NO: 1.

In some embodiments, at least 80% homology or identity comprises: at least 85% homology or identity, at least 90% homology or identity, at least 95% homology or identity, at least 99% homology or identity, or 100% homology or identity. Each possibility represents a separate embodiment of the present invention. Further, and any value or range therebetween of homology or identity to SEQ ID NO: 1 is also possible.

In some embodiments, the isolated bacteria of the invention is a species belonging to the genus Pseudomonas. In some embodiments, the isolated bacteria of the invention is a species belonging to the genus Azotobacter. In some embodiments, the isolated bacteria of the invention is a species belonging to the genus Borreliella.

In some embodiments, the isolated bacteria of the invention has nematode chemotaxis-inducing activity. As used herein, the term “chemotaxis” refers to the movement of an organism (e.g., a nematode) in response to a stimulus. In some embodiments, the isolated bacteria of the invention is a nematode attractant. In some embodiments, the isolated bacteria with at least 80% homology or identity to SEQ ID NO: 1 has nematode chemotaxis-inducing activity. In some embodiments, mutations or alterations may be introduced to the genome of the isolated bacteria of the invention, so long as they do not negatively impact the nematode chemotaxis-inducing activity. In some embodiments, the nematode chemotaxis-inducing activity may be negatively impacted so long as the isolated bacteria remains a stronger attractant than a plant root.

In some embodiments, nematode chemotaxis-inducing activity comprises one or more activities selected from the group consisting of: inducing motility of a nematode, increasing the rate of motility of a nematode, increasing the frequency of motility of a nematode, increasing the number of motile nematodes, increasing the duration of nematode motility, determining the direction of nematode motility, and attracting or luring a nematode.

In some embodiments, the isolated bacteria of the invention has greater nematode chemotaxis-inducing activity than a plant part. In some embodiments, the isolated bacteria of the invention has greater nematode chemotaxis-inducing activity than a plant's root. Non-limiting examples of a plant part include, but are not limited to, a leaf, a stem, a fruit, a root, a shoot, and sap. In some embodiments, the isolated bacteria of the invention has greater nematode chemotaxis-inducing activity than a plant or a part thereof to be protected by a method of the invention.

In some embodiments, greater activity is at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, or 1,000% more activity. Each possibility represents a separate embodiment of the invention. In some embodiments, greater activity is at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, or 1,000% more nematodes attracted. Each possibility represents a separate embodiment of the invention.

In some embodiments, the plant is an eggplant. In some embodiments, the plant is a tobacco plant. In some embodiments, the plant is a plant that can be infected with a nematode. In some embodiments, the plant is a plant that can be attacked by a nematode. In some embodiments, the plant is a plant that attracts nematodes. Examples of plants that can be affected by and attract nematodes include, but are not limited to corn, beans, soybeans, barley, hops, wheat, beats, eggplants, tomato, rice, and tobacco. In some embodiments, a plant part is devoid of the bacteria of the invention. In some embodiments, a plant part is a surface devoid of the bacteria of the invention. In some embodiments, a plant part comprises the bacteria of the invention in an inactivated state. Methods for inactivating bacteria are common and would be apparent to a skilled artisan. Non-limiting examples for methods of bacteria inactivation include, but are not limited to, heating, freezing and thawing, lysis, fragmentation, neutralization, solubilization, and others.

As used herein, the term “nematode” refers to a roundworm belonging to the phylum Nematoda. In some embodiments, the nematode is a root knot nematode. In some embodiments, a nematode belongs to the genus Meloidogyne. In some embodiments, a nematode belonging to the genus Meloidogyne is selected from the group consisting of: M. acronea, M. ardenensis Santos, M. arenaria, M. artiellia, M. brevicauda, M. chitwoodi, M. coffeicola, M. exigua, M. fruglia, M. gajuscus, M. hapla, M. incognita, M. javanica, M. enterolobii (i.e., mayaguensis), M. naasi, M. partityla, and M. thamesi. In one embodiment, a nematode is M. incognita.

### Compositions

According to some embodiments, there is provided a composition comprising an isolated bacteria strain of the genus Pseudomonas, wherein at least 80%, at least 85%, at least 90%, at least 95%, at least 97%, at least 99%, or 100% of the isolated bacteria comprise a genome having at least 95% homology or identity, at least 96% homology or identity, at least 97% homology or identity, at least 98% homology or identity, at least 99% homology or identity, or 100% homology or identity to a genome deposited at NCBI under BioSample accession number SAMN15770455, or any value and range therebetween. Each possibility represents a separate embodiment of the invention.

According to some embodiments, there is provided a composition comprising an isolated bacteria strain of the genus Pseudomonas, wherein 80-90%, 85-95%, 95-97%, 90-98%, 90-99% or 92-100% of the isolated bacteria comprise a genome having 90-99% homology or identity, 91-98% homology or identity, 92-100% homology or identity, 94-98% homology or identity, 91-97% homology or identity, or 95-100% homology or identity, to a genome deposited at NCBI under BioSample accession number SAMN15770455. Each possibility represents a separate embodiment of the invention.

In some embodiments, the present invention is directed to a composition comprising the bacteria of the invention. In some embodiments, at least 70%, 75%, 80%, 85%, 90%, 95%, 97%, 99%, or 100% of the isolated bacteria in the composition is the isolated bacteria of the invention. Each possibility represents a separate embodiment of the invention. In some embodiments, at least 90% of the isolated bacteria in the composition is the isolated bacteria of the invention. In some embodiments, the composition comprises the bacteria of the invention and an acceptable carrier. In some embodiments, the acceptable carrier is an agriculturally acceptable carrier. As used herein an “agriculturally compatible carrier” or “agriculturally acceptable carrier” refers to any material, other than water, which can be added to a plant, a plant part (as disclosed herein above), or a seed thereof without causing or having an adverse effect on the seed (e.g., reducing seed germination) or the plant that grows from the seed, or the like.

In some embodiments, the composition further comprises an agriculturally or environmentally acceptable carrier.

Agricultural carriers may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay.

In some embodiments, the acceptable carrier is an agriculturally suitable and/or environmentally acceptable carrier. Such carriers can be any material that an animal, a plant or the environment to be treated can tolerate. In some embodiments, “environmentally compatible carrier” or “ environmentally acceptable carrier” refers to any material, which can be added to the isolated bacterial strain of the invention, or a composition comprising same (e.g., the composition of the invention) without causing or having an adverse effect on the environment, or any species or an organism other than the nematode inducing plant damage or plant parasitic nematode (PPN), as described herein. Furthermore, the carrier must be such that the composition remains effective at protecting a plant from nematode, e.g., a PPN, at attracting a nematode, e.g., a PPN, or both.

In some embodiments, the composition further comprises a support. In some embodiments, the support is an artificial support. As used herein, the term “artificial support” refers to any man-made material configured to hold or adhere to the bacteria of the invention. In some embodiments, the bacteria maintain the nematode chemotaxis-inducing activity when adhered to or within the support. The support may be configured to have large entry pores and smaller exit pores. The support may be configured to allow entry of a nematode into it and preventing the exit of the nematode. In some embodiments, the support is configured to trap a nematode attracted to the bacteria of the invention. The support may be configured to have a maze architecture or organization. In some embodiments, the entry holes on the outside of the support are large and the inside of the support near the isolated bacteria is small. In some embodiments, the entry holes on the outside of the support are small and the inside of the support near the isolated bacteria are large. Such architectures allow the nematodes to enter due to their attraction to the bacteria and then to have difficulty exiting from the trap. Shapes and architecture of insect, or worm traps, including methods of preparing the same, are well-known in the art.

In some embodiments, the support is a solid support. In some embodiments, the support is a gel. In some embodiments, the gel is a biogel. In some embodiments, the support comprises more than one compartment. In some embodiments, the support is a naturally occurring substance that has been modified. In some embodiments, the modifications are in order to adhere to or contain the isolated bacteria. In some embodiments, the support is naturally occurring, but has artificially been loaded with the isolated bacteria. In some embodiments, the support is a naturally occurring substance that has been isolated. In some embodiments, the support and isolated bacteria of the invention do not occur together in nature.

The support may be made from a variety of materials such as would be suitable for containing or adhering to the bacteria. Examples of these include, but are not limited to, plastic, metal, wood, glass, rubber, charcoal, biochar, shredded plant material, perlite, vermiculite, and coal ash.

In some embodiments, the composition comprises the isolated bacteria of the invention and the support, wherein the isolated bacteria is coupled to the support. In some embodiments, the isolated bacteria is within the support. In some embodiments, the isolated bacteria is reversibly coupled to the support. In some embodiments, the isolated bacteria is irreversibly coupled to the support. In some embodiments, the isolated bacteria cannot diffuse or detach from the support.

As used herein, the term “coupled” encompasses being attached to, adhere to, or both.

In some embodiments, the composition has nematocidal activity. As used herein, the term “nematocidal activity” refers the ability to kill a nematode. In some embodiments, the composition is used as a nematicide. In some embodiments, the nematode starves at the bacteria. In some embodiments, the nematode starves in the composition. In some embodiments, the nematode starves in the trap. In some embodiments, the nematocidal activity comes from the nematode starving upon reaching the composition. In some embodiments, the composition has activity toward a parasitic nematode. In some embodiments, the composition has activity toward a plant-parasitic nematode. In some embodiments, the composition has activity toward a root-knot nematode.

In some embodiments, a composition comprising the bacteria of the invention traps a nematode. In some embodiments, the composition immobilizes a nematode. In some embodiments, the composition prevents or inhibits a nematode from contacting a plant part. In some embodiments, the composition prevents or inhibits a nematode from penetrating to a plant part. In some embodiments, the composition reduces the number of nematodes reaching a plant part. In some embodiments, the composition starves a nematode attracted thereto. In some embodiments, a composition comprising the bacteria of the invention kills a nematode attracted thereto.

In some embodiments, inhibiting is a reduction of at least 5%, at least 15%, at least 25%, at least 35%, at least 50%, at least 75%, at least 85%, at least 90%, at least 95%, at least 100%, or any range or value therebetween, compared to a control. Each possibility represents a separate embodiment of the present invention. In some embodiments, inhibiting by 5-10%, by 7-15%, by 15-25%, by 20-35%, by 30-50%, by 45-75%, by 60-85%, by 70-90%, by 80-95%, or by 90-100%, compared to a control. Each possibility represents a separate embodiment of the present invention. In some embodiments, reducing the number of nematodes is a reduction of at least 5%, 10%, 15%, 20%, 30%, 35%, 40%, 45%, 50%, 55%, 60%, 65%, 70%, 75%, 80%, 85%, 90%, 95%, 97%, 99%, 100%, or any range or value therebetween, in the number of nematodes reaching a plant part. Each possibility represents a separate embodiment of the invention.

In one embodiment, a control comprises a composition comprising an inactivated form of the bacteria of the invention, as disclosed hereinabove.

In some embodiments, a composition comprising the isolated bacteria of the invention or a method of use thereof reduce the survival of a nematode. In some embodiments, the composition or method of use thereof reduce nematode survival by at least 5%, by at least 15%, by at least 25%, by at least 35%, by at least 50%, by at least 75%, by at least 85%, by at least 90%, by at least 95%, by at least 99%, by at least 100%, or any range or value therebetween, compared to a control. In some embodiments, the composition or method of use thereof reduce nematode survival by 5-10%, by 7-15%, by 15-25%, by 20-35%, by 30-50%, by 45-75%, by 60-85%, by 70-90%, by 80-95%, or by 90-100%, compared to a control. Each possibility represents a separate embodiment of the present invention.

In some embodiments, the composition further comprises one or more nematode attractants. Non-limiting examples of nematode attractants include, but are not limited, to cyclic nucleotides (e.g., cAMP and cGMP), anions (e.g., Cl−, Br−, and I−), cations (e.g., Na+, Li+, K+, Mg2+, and alkaline pH values. In some embodiments, the nematode attractant is not biological. In some embodiments, the nematode attractant is not an organism.

In some embodiments, the composition further comprises a nematode killing molecule. In some embodiments, the nematode killing molecule is selected from the group consisting of: a polypeptide, a polynucleotide (such as an RNA polynucleotide), a chemical, and a small molecule. Nematode poisons are well known in the art and may be incorporated into the compositions of the invention to enhance their efficacy in protecting a plant. Alternatively, as such poisons are often toxic to other organisms, including humans, the compositions of the invention are of extreme benefit as they function without such additional toxins.

### Methods of Use

According to some embodiments, there is provided a method for attracting a nematode to a surface, comprising contacting the surface with bacteria having nematode chemotaxis-inducing activity, thereby attracting the nematode to the surface.

As used herein, the term “surface” encompasses any area or material onto which bacteria can be deposited. In some embodiments, the surface is a natural surface. In some embodiments, the surface is a man-made or artificial surface.

According to some embodiments, there is provided a method for protecting a plant from nematode-induced damage, the method comprising: contacting a growth medium comprising a nematode with a composition comprising bacteria having a nematode chemotaxis-inducing activity and a support, wherein the bacteria is coupled to or within the support, thereby protecting the plant from nematode-induced damage.

According to some embodiments, there is provided a method for reducing the pathogenicity of a plant parasitic nematode (PPN), comprising contacting a growth medium comprising the PPN with an effective amount of the isolated bacterial strain of the invention, or a composition comprising same.

In some embodiments, reducing the pathogenicity of the PPN comprises reducing the survival of the PPN.

In some embodiments, reducing the survival comprises directly or indirectly reducing the survival. In some embodiments, directly reducing the survival comprises killing or destroying the PPN. In some embodiments, indirectly reducing the survival comprises not directly predating, killing or destroying the PPN but rather indirectly reduce their survival or viability, e.g., by starvation due to entrapment in an artificial solid support, as disclosed herein.

In some embodiments, reducing the pathogenicity of the PPN comprises: reducing the average number of eggs, cysts, or both, of the PPN per weight of a plant or a part thereof, reducing the root galling index of the root of the plant, reducing the penetration rate of the PPN to the plant or a part thereof, reducing the number of the PPN penetrating the plant or a part thereof, or any combination thereof.

As used herein, protecting a plant from nematode-induced damage comprises treating a plant afflicted by nematode-induced damage, preventing nematode-induced damage in a plant, or both.

As used herein, the term “treating” a nematode-induced damage or disease, encompasses alleviation of at least one symptom thereof, a reduction in the severity thereof, or inhibition of the progression thereof. Treatment need not mean that the disease, or damage is totally cured. To be an effective treatment, a useful composition herein needs only to reduce the severity of a disease, or damage, reduce the severity of symptoms associated therewith, or provide improvement to a plant's wellbeing. In some embodiments, treating nematode-induced damage comprises attracting a nematode in the plant away from the plant.

As used herein, the term “prevention” of nematode-induced damage or disease encompasses the delay, prevention, suppression, or inhibition of the onset of a disease, or damage. As used in accordance with the presently described subject matter, the term “prevention” relates to a process of prophylaxis in which a plant or a part thereof is grown in growth media that is exposed to the presently described composition prior to the induction or onset of the disease/damage. The term “suppression” is used to describe a condition wherein the disease/damage has already begun but obvious symptoms of the condition have yet to be realized. In either case, the term prophylaxis can be applied to encompass both prevention and suppression. In some embodiments, preventing nematode-induced damage comprises attracting a nematode in the growth media away from the plant.

In some embodiments, protecting a plant according to the disclosed method comprises reducing the penetration rate of a nematode to the root of the plant, reducing the number of nematodes penetrating the root, reducing the number of hatching eggs of the nematode, reducing the egg hatching rate of the nematode, attracting a nematode to a composition comprising the bacteria of the invention, killing a nematode, or any combination thereof.

As used herein, the term “growth medium” refers to any solid, liquid or semi-solid designed to support the growth of a plant. In some embodiments, growth medium is soil or dirt. In some embodiments, the growth medium comprises a nematode. In some embodiments, the growth media is infested with nematodes. In some embodiments, the growth medium is suitable to support a nematode.

In some embodiments, the plant is growing in the growth medium. In some embodiments, at least a root of a plant is growing in the growth medium. In some embodiments, a root of plant is growing in the growth medium. In some embodiments, the root of a plant protected according to the disclosed method is within the growth medium.

In some embodiments, the composition of the invention is a greater attractant of a nematode that is the plant or a root of the plant. In some embodiments, the composition of the invention is at least twice as good an attractant as the plant or root of the plant. In some embodiments, the composition of the invention is at least ten times as good an attractant as the plant or root of the plant. A skilled artisan will appreciate that by attracting the nematodes to the composition they are kept away from the plant and thus keep the plant free of or reduce the damage caused by nematodes. Unlike, nematode killing bacteria that would be placed on the plant itself, or its roots the composition of the invention is placed at a distance from the plant to pull the nematodes away from the plant.

In some embodiments, protecting a plant according to the disclosed method is by contacting the growth medium used for culturing the plant with the composition of the invention. In some embodiments, contacting the growth medium does not comprise contacting the root or the plant. In some embodiments, contacting is at a distance sufficient to keep attracting nematodes to the composition of the invention and not the plant. In some embodiments, contacting the growth medium is at a distance of at least 0.1 cm, at least 0.2 cm, at least 0.5 cm, at least 1 cm, at least 5 cm, at least 10 cm, at least 50 cm, at least 1 m, at least 1.5 m, at least 2 m from the root of the plant. Each possibility represents a separate embodiment of the invention. In some embodiments, contacting the growth medium is at a distance of 0.1-0.5 cm, 0.4-1 cm, 0.5-5 cm, 1-15 cm, 5-50 cm, 40-100 cm, 50-150 cm, or 1-2 m from the root of the plant. Each possibility represents a separate embodiment of the invention. In some embodiments, contacting the growth medium is at a distance of at most 5 cm, 10 cm, 20 cm, 30 cm, 40 cm, 50 cm, 60 cm, 70 cm, 80 cm, 90 cm, 100 cm, 150 cm, 200 cm, 250 cm, 300 cm, 400 cm, 500 cm, 600 cm, 700 cm, 750 cm, 800 cm, 900 cm, 1,000 cm, 1,500 cm, 2,000 cm, 2,500 cm, 3,000 cm, 3,500 cm, 4,000 cm, 4,500 cm, or 5,000 cm, or any range or value therebetween. Each possibility represents a separate embodiment of the invention. In some embodiments, contacting the growth medium is at a distance of 2-5 m at most.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as weight, is to be understood to include any integer within the recited range, unless otherwise indicated.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Materials and methods

### Bacterial Isolates

The root system of an eggplant infested with Meloidogyne incognita was collected from the Yair R&D station in Hatzeva in May 2018. The roots were washed with ultrapure water and were soaked in sodium hypochlorite 1% (v/v) for 5 minutes. After additional wash with ultrapure water, mature knots were scraped externally with a sterilized scalpel, dissected and placed in 15 ml Phosphate Buffered Saline (PBS; Biological Industries) and stirred vigorously. Forty (40) μl of the PBS were then smeared on a beef-extract-peptone agar (BEPA) plate (beef extract 3 g/L; peptone 10 g/ L; NaCl 5 g/L; agar 20 g/L; pH 7.0), which was incubated for 72 hours at 37 ° C. under anoxic conditions (5 L modular incubator chamber (billups-rothenberg) with two 2.5 L AnaeroGen atmosphere generation sachets (Thermo Scientific)), together with a blank agar-BEPA plate to verify the sterility of the medium. After 72 hours, one colony of each unique colony form detected was isolated on a fresh BEPA plate, and the plates were again incubated for additional 72 hours under the same conditions. Isolated pure clones were scooped into 5 ml of liquid beef-extract-peptone medium and left to incubate for additional 72 hours. The liquid cultures were further used as described herein below. All bacterial manipulations were carried out in a type 2 biological hood (MRC).

### Phase 2 Juvenile (J2) Isolation

To isolate J2 larvae for each attraction assay in a 12-well plate, the root systems of three eggplants was thoroughly washed with ultrapure water, chopped with a scalpel and placed in a bearman tray overnight, as previously described (Williamson and Cepulyte (2017)). The bearman tray filtrate was then filtered through No. 1 Whatman paper. The J2 were resuspended in 1 ml of ultrapure water and the purity of the J2 community was inspected under a dissecting microscope. J2 of Meloidogyne are at least 3 times smaller than those of other nematodes, which enabled to confirm that the extracted community comprised Meloidogyne only.

### Bacterial Isolate Identification

For taxonomy identification, DNA was extracted from the isolate colonies using the DNeasy blood and tissue DNA extraction kit (Qiagen) and the 16S-rRNA gene was amplified by PCR reaction using the primers BacSSU_FAM27f (5′-GAGTTTGATCMTGGCTCAG-3′; SEQ ID NO: 2) and BacSSU_1407R (5′-GACGGGCGGTGTGTRC-3′; SEQ ID NO: 3). The PCR reaction (one cycle at 95° C.-3 min; 33 cycles at 98° C.-20 s, 57° C.-15 s, 72° C.-21 s; one cycle at 72° C.-1 min) was carried out in triplicate with the KAPA HiFi HotStart ReadyMix PCR Kit (Kapa Biosystems) following the provided instructions, on a SimpliAmp thermal cycler (ABI). PCR products were purified with Agencourt AMPure XP and directly sequenced on an ABI PRISM BigDye Terminator sequencer. The 1,380 bp long amplification product was subjected to the online blast analysis using the whole blast nucleotide database.

### Attraction Assays

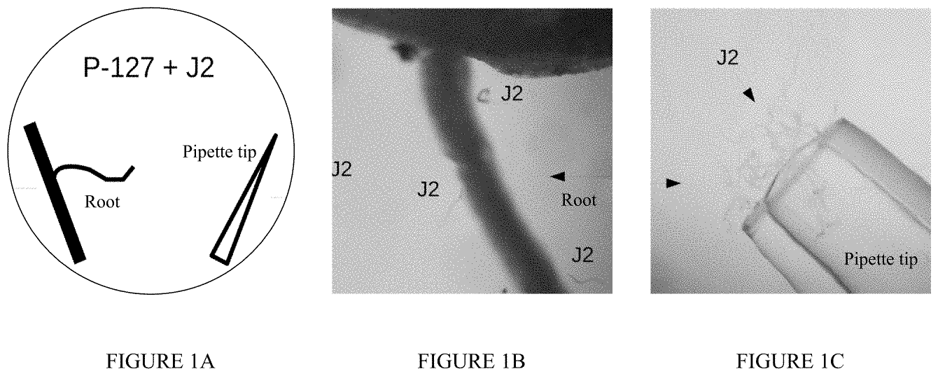

Attraction assays were carried out in two 12-well plates with each well comprising 1 ml Pluronic P-127 Tris MES buffer gel, prepared as previously described (Williamson and epulyte (2017)), and mixed with approximately 200 J2 larvae. The isolate contained by a pipette tip was placed in the well with an eggplant root fragment, 20 mm apart from one another and the number of attracted J2 larvae to each was compared (FIG. 1). This was performed in eight replicates for each bacterial isolate tested. For control, the root fragment was placed in a well together with a pipette tip containing sterile BEPA medium, and attraction was compared as mentioned above. The assay lasted for 10 minutes, after which all J2 larvae reaching the rhizoplane were counted as well as J2 larvae which aggregated in the bacteria-containing pipette tip.

### Egg Hatching Inhibition Assay

To test egg hatching inhibition by the bacterial secretions, a single egg mass was placed in the middle of each well in a 12-well plate, within 1 ml of pluronic gel. Eggs were exposed to four treatments: (1) Biocasle capsule (Menashe and Kurzbaum, 2014) containing double distilled water (DDH), (2) Biocasle capsule containing the sterile medium, (3) Biocasle capsule containing the filtered medium (see Attraction assay 2), and (4) Biocasle capsule containing the bacteria in their medium (see Attraction assay 2). Each treatment was replicated across six wells. Hatchlings were counted after 24 hours and 48 hours.

### Example 1

Bacterial Isolate 1 Attracts J2 Larvae

Six unique colony forms were identified in the isolation process, two of which appeared to be bacterial, rather than fungal. The inventors denoted them isolates 1 and 6 and further tested them in the attraction assays. In the attraction assays, the root fragments attracted 5 to 17 J2 larvae, when co-incubated with isolate 6 or control (containing no bacterial isolates). In all replicates containing isolate 1, more than 70 J2 larvae were attracted to the bacteria-containing tip within the inspected timeframe. No J2 larvae were observed to be attracted to a pipette tip containing either isolate 6 or a sterile medium. Assay results are summarized herein below (Table 1).

TABLE 1
First attraction assay - results

| | Attraction to root | Attraction to tip |
|---|---|---|
| Trial | mean (min, max) | mean (min, max) |
| Root + isolate 1 | 8.25 (6, 11) | 90 (70,120) |
| Root + isolate 6 | 7.25 (5, 10) | 0 |
| Root + empty pipette tip | 9.9 (7, 13) | 0 |

A second attraction assay was designed to compare the attraction of the isolate, with that of its filtrate. To produce a filtrate, the bacterium was cultured in BEPA medium for three days, as described above. The medium was then filtered through a 0.45 micron pore-size filter. The assay was carried out as above, using basil roots, with 4 treatments in 4 replicates each: (i) root vs. the isolate, (ii) root vs. the filtrate, (iii) root vs. the sterile medium and (iv) root only. The assay plate was incubated overnight, after which, the roots were stained following (as in Thies et al. (2002)), and the J2s on the roots and tips were counted using a dissection microscope. The results are summarized in Table 2.

TABLE 2
Second attraction assay 2 - results

| | | |
|---|---|---|
| | Attraction to root | Attraction to tip |
| Trial | mean (min, max) | mean (min, max) |

| | | | | |
|---|---|---|---|---|
| Root + isolate 1 | 8 | (4, 11) | 20 | (12, 35) |
| Root + isolate 1 filtrate | 7.33 | (5, 7) | 19.25 | (1, 66) |
| (no bacteria) | | | | |
| Root + sterile medium | 9.67 | (4, 15) | 9.25 | (1, 14) |

| | | | |
|---|---|---|---|
| Root only | 22.5 | (19, 26) | — |

According to the results, both the filtrate and the bacterium were at least twice as attractive for the J2s than the root fragment. This indicates that an active compound, which is secreted from the bacterium, can be utilized independently.

### Example 2

### Bacterial Isolate 1 is a New and Distinct Pseudomonas Species

A 16S-rRNA gene was amplified by PCR using DNA extract of bacterial isolate 1 as a template and a set of primers as mentioned above. The product was purified and sequenced, and subsequently bioinformatically analyzed for sequence homology.

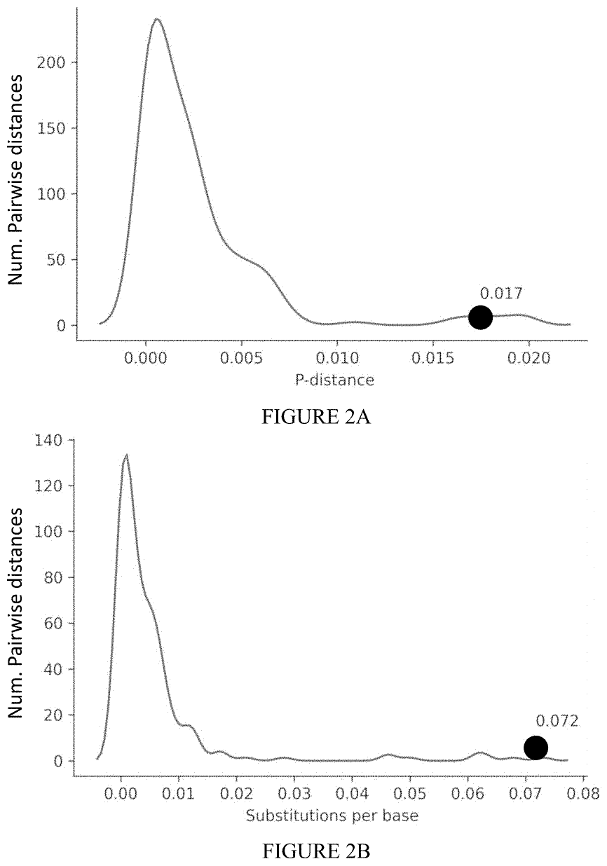

To determine whether this isolate is a new species, the 318 most similar sequences were retrieved from GenBank using the online version of BLAST. These sequences represented 220 species. The sequences were aligned using MAFFT with 1,000 maximum iterations, and the resulting alignment was trimmed with the gappyout algorithm in TrimA1. A phylogenetic tree was reconstructed with FastTree 2.1, with the GTR substitution model. This data was used to compute a distribution of intra-specific distances (FIG. 2), based on either the proportion of divergent positions (FIG. 2A) or tree-branch distances (FIG. 2B). Both methods revealed the distance between the isolate and its closest sequenced relative (GenBank accession NR_151929.1) is significantly larger than that expected within a species (P value<0.0227 and P value<0.0045, respectively). Therefore, the newly isolated bacteria is a novel species comprising a SSU sequence which is 1.7% divergent from its closest relative.

Further, the inventors have performed a broad genetic distance analysis. To compute the genetic distance between the genome assembly of the new bacterial isolate of the invention and the closest published genome, the inventors queried the NCBI RefSeq genome database with SEQ ID NO: 1 as the query, using the online version of blastn. The best match was found in genome entry CP016162, which was then downloaded completely and aligned to the scaffolds of the herein disclosed isolate's genome assembly using the program Mummer4. All scaffolds longer than 10,000 bp were included and a cumulative length of 4,230,689 bp was successfully aligned. The weighted average identity between the scaffolds and the reference sequence was 93%.

In summary, the inventors had isolated and identified a new and distinct species of Pseudomonas, found in eggplant knots, which is an order of magnitude more attractive to root knot nematode J2 larvae than the eggplant root itself, and suggest it as a candidate for biological control use.

### Example 3

### Bacteria and Secretion of Same Inhibit the Hatching of Pathogenic Nematode Eggs

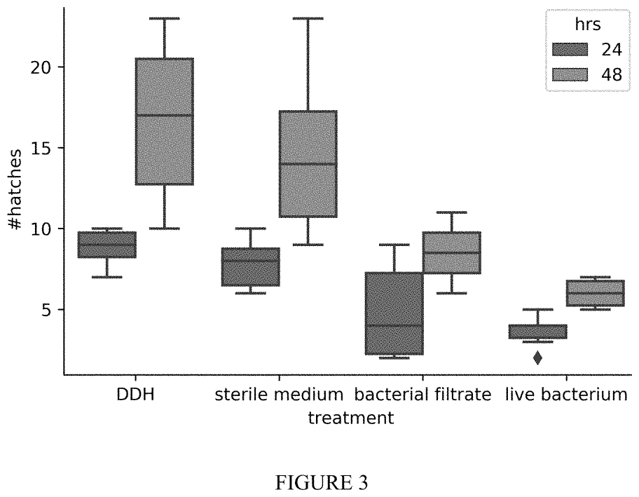

The inventors further examined whether the bacteria per se, or secretion thereof, inhibit the hatching of a pathogenic nematode eggs. Indeed, the inventors showed that after 24 hours of incubation, a significant reduction in hatching was observed with the live bacteria, compared with controls (e.g., DDH or sterile medium treatments, FIG. 3 (corrected p-value<0.01)). After 48 hours, a significant reduction in hatching was observed with the live bacteria and with the bacterial filtrate, both compared with the relevant controls (e.g., DDH and sterile medium treatments, with a corrected p-value<0.01 and <0.03, respectively (see FIG. 3).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. (canceled)

2. (canceled)

3. (canceled)

4. (canceled)

5. (canceled)

6. (canceled)

7. A composition comprising a bacterial strain of the genus Pseudomonas comprising a genome having at least 94% homology or identity to a genome deposited at NCBI under BioSample accession number SAMN15770455, and an agriculturally or environmentally acceptable carrier.

8. The composition of claim 7, further comprising an artificial support, optionally wherein said isolated bacterial strain is coupled to or within said artificial support, and optionally , wherein said artificial support is configured to trap a nematode attracted to said isolated bacterial strain, optionally wherein said nematode belongs to the genus Meloidogyne, and optionally wherein said nematode is Meloidogyne incognita.

9. (canceled)

10. (canceled)

11. (canceled)

12. (canceled)

13. A method for attracting a nematode to a surface, comprising contacting said surface with bacteria having nematode chemotaxis-inducing activity, thereby attracting said nematode to said surface.

14. A method for protecting a plant from nematode-induced damage, comprising: contacting a growth medium comprising a nematode with a composition comprising bacteria having a nematode chemotaxis-inducing activity and an artificial support, wherein said bacteria is coupled to or within said artificial support, thereby protecting said plant from nematode-induced damage.

15. The method of claim 14, wherein said bacteria belong to the genus Pseudomonas.

16. (canceled)

17. (canceled)

18. The method of claim 14, wherein said nematode is a plant parasitic nematode (PPN), optionally said nematode belongs to the genus Meloidogyne, and optionally wherein said nematode belonging to the genus Meloidogyne is M. incognita.

19. (canceled)

20. (canceled)

21. The method of claim 14, wherein said composition comprises a bacterial strain of the genus Pseudomonas comprising a genome having at least 94% homology or identity to a genome deposited at NCBI under BioSample accession number SAMN15770455, and an agriculturally or environmentally acceptable carrier.

22. The method of claim 14, wherein said composition is more attractant of said nematode than said root is attractant of said nematode, and optionally wherein said composition is at least twice as attractant as said root.

23. (canceled)

24. The method of claim 14, wherein said protecting said plant comprises reducing the penetration rate of said nematode to said root, reducing the number of nematodes penetrating said root, reducing the number of hatching eggs of said nematode, reducing the egg hatching rate of eggs of said nematode, or any combination thereof.

25. The method of claim 14, wherein said bacteria having nematode chemotaxis-inducing activity traps said nematode in said composition, and optionally wherein said composition reduces the survival of said nematode.

26. (canceled)

27. The method of claim 14, wherein said composition kills said nematode.

28. The method of claim 14, wherein said artificial support is configured to trap a nematode attracted to said bacteria.

29. The method of claim 14, wherein said composition has nematocidal activity.

30. The method of claim 14, wherein said protecting comprises attracting said nematode to said composition.

31. The method of claim 14, wherein said contacting said growth medium does not comprise contacting a root of said plant, and optionally wherein said contacting said growth medium is at a distance of at least 0.1 cm from a root of said plant.

32. (canceled)

33. The composition of claim 7, wherein said bacterial strain comprises a genome having 100% homology or identity to said genome deposited at NCBI under a BioSample accession number SAMN15770455.

34. The composition of claim 7, wherein said bacterial strain comprises a polynucleotide sequence having at least 90% homology or identity to SEQ ID NO: 1.

35. The composition of claim 34, wherein said bacterial strain comprises a polynucleotide sequence having 100% homology or identity to SEQ ID NO: 1.

36. The composition of claim 34, wherein said polynucleotide sequence having at least 90% homology or identity to SEQ ID NO: 1 is a 16S-rRNA sequence.

37. The composition of claim 7, wherein said bacterial strain is characterized by having a nematode chemotaxis-inducing activity, a nematode egg hatching inhibiting activity, a nematocidal activity, or any combination thereof.
